(19) United States
(12) Patent Application Publication
(10) Pub. No.: US 2026/0294836 A1
(43) Pub. Date: Oct. 1, 2026

(54) REMOTE CONTROL AND QUANTITATIVE MONITORING OF DRUG RELEASE FROM NANOPARTICLES BASED ON MAGNETIC PARTICLE IMAGING

(71) Applicant: Board of Trustees of Michigan State University, East Lansing, MI (US)

(72) Inventors: Bryan SMITH, East Lansing, MI (US); Yapei ZHANG, East Lansing, MI (US)

(21) Appl. No.: 18/998,182

(22) PCT Filed: Jul. 27, 2023
(86) PCT No.: PCT/US2023/028888

§ 371 Date: Jan. 24, 2025

Related U.S. Application Data
(60) Provisional application No. 63/393,278, filed on Jul. 29, 2022.

(51) Int. Cl. A61K 9/51; A61K 9/00; A61K 31/704; G06T 7/00

(52) U.S. Cl. CPC A61K 9/5192; A61K 9/0019; A61K 9/5115; A61K 9/513; A61K 31/704; G06T 7/00

(57) ABSTRACT

A method for targeting and tuning drug release in a human or non-human subject includes monitoring release of one or more drugs from one or more nanocomposites administered to the subject and applying precision-controlled optical stimulus to an area of the subject hosting the one or more nanocomposites to accelerate release of the one or more drugs from the one or more nanocomposites.

### CROSS-REFERENCE TO RELATED APPLICATIONS

This application claims the benefit of U.S. Provisional Application No. 63/393,278 filed Jul. 29, 2022. The entire disclosure of the above application is incorporated herein by reference.

### FIELD

The present disclosure relates to methods for monitoring, maintaining, and adjusting already-administered drug doses within specific therapeutic ranges.

### BACKGROUND

This section provides background information related to the present disclosure which is not necessarily prior art.

Many commonly used cancer drugs, such as doxorubicin (“DOX”), are toxic to normal tissues and are functional only within a narrow concentration range known as the “therapeutic window.” The therapeutic window represents a dose range between minimum effective drug dose (“MED”) and the minimum toxic drug dose (“MTD”). Drug concentrations should constantly remain between the MED and the MTD to optimize therapeutic effects while minimizing adverse effects. The clinically-injected dosage for chemotherapy treatment is often computed based on body surface area, which is an overly simplistic one-size-fits-all-type strategy that results in widely heterogeneous patient responses. In general, administration of too much drug (i.e., above the therapeutic window) can cause serious side effects on normal human organs, while too little drug (i.e., below the therapeutic window) can results in lack of treatment efficacy and may result in drug-induced cancer drug resistance, which is a primary cause of chemotherapy failure.

Intratumoral drug concentrations are often unknown and the lack of sufficient drug concentrations only apparent when a key outcome metric is measured, such as tumor size. Drug release monitoring (“DRM”) approaches, including those using, for example, magnetic resonance imagining (“MRI”), optical imaging, and photoacoustic imaging, are emerging that can provide the ability to quantify drug release rates and thus drug concentrations in a region over time. However, despite some promise, these modalities often display issues undermining their clinical translation. For example, while MRI displays excellent penetration depth, it is often not linearly quantitative and intrinsic/background signal can convolute drug distribution signals, decreasing quantitative reliability. Optical imaging and photoacoustic imaging are often limited by penetration depth and signal convolution. Magnetic particle imaging (“MPI”) is a promising DRM approach that does not have the drawbacks of MRI, optical imagining, and/or photoacoustic imaging. MPI is a non-invasive imaging modality the employs superparamagnetic nanoparticles (“NPs”) as contrasting agents and offers excellent imaging depth, linearly quantifiable signal, and real-time imaging capability. MPI is also sensitive to contrast agent's local nanoenvironment. For at least these reasons, MPI has broad potential for quantitative monitoring of drug release in vivo at large depths.

While DRM approaches (like MPI) provides important information on local dose concentrations, it alone is insufficient to maintain drug concentrations in the therapeutic window because it cannot adjust said dose. For example, if by using a DRM approach it is determined that the dose is insufficient (i.e., below the therapeutic window), more of the drug(s) would be required to enter the therapeutic window. It would be desirable to combine quantifying approaches, like DRM approaches, and remote-controlled actuations (for example, near-infrared (NIR) laser light) to provide improve methods for monitoring, maintaining, and adjusting administered doses within specific therapeutic ranges in real time while limiting off-target toxicities.

### SUMMARY

This section provides a general summary of the disclosure and is not a comprehensive disclosure of its full scope or all of its features.

In various aspects, the present disclosure provides a method for targeting and tuning in vivo drug release in a human or non-human subject. The method may include monitoring release of one or more drugs from one or more nanocomposites administered to the subject and applying precision-controlled optical stimulus to an area of the subject hosting the one or more nanocomposites to accelerate release of the one or more drugs from the one or more nanocomposites.

In at least one example embodiment, the monitoring may include collecting information regarding the release of the one or more drugs from the one or more nanocomposites, and the method may further include analyzing the collected information to determine if a concentration of the released one or more drugs is within a therapeutic window; if the concentration of the released one or more drugs is below the therapeutic window, initiating the application of the precision-controlled optical stimulus; and if the concentration of the released one or more drugs is within the therapeutic window, continuing to monitor the release of the one or more drugs from the one or more nanocomposites.

In at least one example embodiment, the monitoring may include magnetic particle imaging, where the intensity of magnetic particle imaging signals varies in response to the release of the one or more drugs.

In at least one example embodiment, the precision-controlled optical stimulus may include near-infrared light irradiation.

In at least one example embodiment, the precision-controlled optical stimulus may be applied for a period greater than or equal to about 1 minute to less than or equal to about 20 minutes.

In at least one example embodiment, the one or more nanocomposites may each include a biodegradable polymer shell that at least partially encompasses a core that includes magnetic nanoparticles.

In at least one example embodiment, the biodegradable polymer shell may include a polymeric material selected from the group consisting of: poly(lactide-co-glycolide acid) (PLGA), polymerized ursodeoxycholic acid (PUDCA), polyethylenimine (PEI), chitosan, poly(d-lactic acid), poly(s-caprolactone) (PCL), polylactic acid, polyanhydrides, polyphosphoesters, polyurethanes, poly(caprolactone), hyaluronic acid, albumin, polysaccharides, and combinations thereof.

In at least one example embodiment, the magnetic nanoparticles may include Fe₃O₄ nanoparticles.

In at least one example embodiment, the one or more drugs may be dispersed with the magnetic nanoparticles in the core.

In at least one example embodiment, the one or more drugs may be embedded in the biodegradable polymer shell.

In at least one example embodiment, the one or more drugs may include doxorubicin.

In at least one example embodiment, the method may further include preparing the one or more nanocomposites by contacting nanoclusters including the biodegradable polymer shell and the magnetic nanoparticles with the one or more drugs.

In at least one example embodiment, the method may further include preparing the one or more nanoclusters by contacting the magnetic nanoparticles and the biodegradable polymer in a solvent and applying a mixing force.

In at least one example embodiment, the method may further include administering the one or more nanocomposite to the subject.

In various aspects, the present disclosure provides another method for targeting and tuning in vivo drug release in a human or non-human subject. The method may include collecting information regarding release of one or more drugs from one or more nanocomposites administered to a subject; determining, using the collected information, if a concentration of the released one or more drugs is outside of a therapeutic window; if the concentration of the released one or more drugs is within the therapeutic window, continuing to collect information regarding the release of the one or more drugs from the one or more nanocomposites; and if the concentration of the released one or more drugs is below the therapeutic window, causing a precision-controlled optical stimulus to be applied to an area of the subject hosting the one or more nanocomposites for a period greater than or equal to about 1 minute to less than or equal to about 20 minutes to accelerate the release of the one or more drugs from the one or more nanocomposites.

In at least one example embodiment, the monitoring may include magnetic particle imaging, where the intensity of magnetic particle imaging signals varies in response to the release of the one or more drugs, and the precision-controlled optical stimulus including near-infrared light irradiation.

In at least one example embodiment, the one or more nanocomposites may each include a biodegradable polymer shell that at least partially encompasses a core that includes magnetic nanoparticles, where the one or more drugs are dispersed with the magnetic nanoparticles within the core, embedded in the biodegradable polymer shell, or a combination thereof.

In at least one example embodiment, the method may further include preparing the one or more nanocomposites by contacting nanoclusters including the biodegradable polymer shell and the magnetic nanoparticles with the one or more drugs.

In at least one example embodiment, the method may further include administering the one or more nanocomposite to the subject.

In various aspects, the present disclosure provides a method for targeting and tuning in vivo drug release in a human or non-human subject. The method may include collecting information regarding release of one or more drugs from one or more nanocomposites administered to a subject using magnetic particle imaging, where the intensity of magnetic particle imaging signals varies in response to the release of the one or more drugs; determining, using the collected information, if a concentration of the released one or more drugs is below the therapeutic window; if the concentration of the released one or more drugs is within the therapeutic window, continuing to collect information regarding the release of the one or more drugs from the one or more nanocomposites; and if the concentration of the released one or more drugs is outside of the therapeutic window, using near-infrared light irradiation to accelerate the release of the one or more drugs from the one or more nanocomposites, the near-infrared light irradiation applied to an area of the subject hosting the one or more nanocomposites.

Further areas of applicability will become apparent from the description provided herein. The description and specific examples in this summary are intended for purposes of illustration only and are not intended to limit the scope of the present disclosure.

### DRAWINGS

The drawings described herein are for illustrative purposes only of selected embodiments and not all possible implementations and are not intended to limit the scope of the present disclosure.

FIG. 1 is a flowchart illustrating an example method for targeting and tuning drug release or application in accordance with various aspects of the present disclosure.

FIG. 2A is a scanning electron microcopy image of a superparamagnetic nanoclusters with a transmission electron microscopy image insert of the superparamagnetic nanoclusters, where the superparamagnetic nanoclusters is prepared in accordance with various aspects of the present disclosure;

FIG. 2B is a scanning electronic microscopy image of drug-loaded superparamagnetic nanocomposites with a transmission electron microscopy image insert of drug-loaded superparamagnetic nanocomposites, where the drug-loaded superparamagnetic nanocomposites is prepared in accordance with various aspects of the present disclosure;

FIG. 2C is a UV-Vis adsorption spectra of superparamagnetic nanoclusters and drug-loaded superparamagnetic nanocomposites, where the superparamagnetic nanoclusters and drug-loaded superparamagnetic nanocomposites are prepared in accordance with various aspects of the present disclosure;

FIG. 2D is a dynamic light scatting spectra of superparamagnetic nanoclusters and drug-loaded superparamagnetic nanocomposites, where the superparamagnetic nanoclusters and drug-loaded superparamagnetic nanocomposites are prepared in accordance with various aspects of the present disclosure;

FIG. 3A is a graphical demonstration illustrating a relationship between doxorubicin release and magnetic particle imaging signal intensity for drug-loaded superparamagnetic nanocomposites prepared in accordance with various aspects of the present disclosure and dispersed in a phosphate buffered saline solution having a pH of 6.5 to simulate tumor acidic microenvironment;

FIG. 3B is a graphical demonstration illustrating a relationship between doxorubicin release and magnetic particle imaging signal intensity in a 10% serum having a pH of 6.5 to simulate blood plasma;

FIG. 3C is a graphical demonstration illustrating a relationship between doxorubicin release and magnetic particle imaging signal intensity for 4T1 cells demonstrating that drug release may be monitored in the 4T1 cells;

FIG. 3D is a graphical demonstration illustrating a relationship between doxorubicin release and magnetic particle imaging signal intensity for drug-loaded superparamagnetic nanocomposites prepared in accordance with various aspects of the present disclosure and dispersed in a phosphate buffered saline solution having a pH of 7.4 to simulate blood pH demonstrating that drug-loaded superparamagnetic nanocomposites is stable in blood;

FIG. 4A are merged magnetic particle imaging and CT scans for tumor-bearing mice injected intratumorally with drug-loaded superparamagnetic nanocomposites prepared in accordance with various aspects of the present disclosure and stimulated with near infrared for different time periods in accordance with various aspects of the present disclosure;

FIG. 4B is a graphical demonstration illustrating a relationship between time of near-infrared stimulations and magnetic particle imaging signal increases;

FIG. 4C is a graphical demonstration illustrating a relationship between time of near-infrared stimulations and doxorubicin release;

FIG. 5A is a graphical demonstration illustrating a relationship between doxorubicin release and magnetic particle imaging signal intensity for drug-loaded superparamagnetic nanocomposites prepared in accordance with various aspects of the present disclosure and dispersed in a phosphate buffered saline solution having a pH of 7.4 to simulate blood pH, when near infrared-stimulated drug release occurs upon 1 minute, 3 minutes, 5 minutes, and 7 minutes of NIR illumination;

FIG. 5B is a graphical demonstration illustrating a relationship between doxorubicin release and magnetic particle imaging signal intensity for 4T1 cells, when NIR-stimulated drug release occurs upon 1 minute, 3 minutes, 5 minutes, and 7 minutes of NIR illumination, demonstrating that drug release can be monitored in the 4T1 cell line upon NIR illumination;

FIG. 5C is a scanning electronic microscopy image of drug-loaded superparamagnetic nanocomposites with a transmission electron microscopy insert of drug-loaded superparamagnetic nanocomposites, where the drug-loaded superparamagnetic nanocomposites is prepared in accordance with various aspects of the present disclosure and subjected to instances of optical near infrared-based energy, demonstrating that drug-loaded superparamagnetic nanocomposites can be degraded using NIR illumination;

FIG. 5D includes graphical demonstrations illustrating flow cytometric apoptosis assay for different samples as prepared in accordance with various aspects of the present disclosure for different cell types;

FIG. 6A is a graphical demonstration quantifying bioluminance signal intensity in 4T1 breast tumor after different treatments prepared in accordance with various aspects of the present disclosure, where bioluminance signal decreases when the 4T1 tumor cells are dying;

FIG. 6B is a graphical demonstration quantifying signal intensity in the 4T1 breast tumor 15 days of the different treatments;

FIG. 6C is a graphical demonstration illustrating tumor growth in subjects receiving different samples prepared in accordance with various aspects of the present disclosure;

FIG. 6D is a graphical demonstration illustrating apoptosis is subjects receiving different samples prepared in accordance with various aspects of the present disclosure;

FIG. 6E are transmission electron microscopy images of tumor sections treated with different samples prepared in accordance with various aspects of the present disclosure;

FIG. 6F are immunochemistry images of tumor sections treated with different samples prepared in accordance with various aspects of the present disclosure;

FIG. 6G are tunnel assays of tumor sections treated with different samples prepared in accordance with various aspects of the present disclosure;

FIG. 6H are Prussian blue staining of tumor sections treated with the different samples prepared in accordance with various aspects of the present disclosure;

FIG. 7A is a graphical demonstration comparing 4T1 tumor (DOX-sensitive) volumes for subjects treated with different samples prepared in accordance with various aspects of the present disclosure;

FIG. 7B is a graphical demonstration comparing BT549 tumor (DOX-resistant) volumes for subjects treated with different samples prepared in accordance with various aspects of the present disclosure, where the inset is a graphical demonstration the 10 days SPNCD+NIR 7 min treated mice;

FIG. 7C are BLI images of subjects (4T1 tumor bearing Balb/c mice) receiving different samples prepared in accordance with various aspects of the present disclosure, where the PBS is thirty-days post injection, the DOX is 60 days post intratumoral injection, and SPNCD+NIR treated is 75 days post intratumoral injection;

FIG. 7D is a graphical demonstration comparing body weights of subjects treated with different samples prepared in accordance with various aspects of the present disclosure;

FIG. 7E is a graphical demonstration comparing LDH activity at 60 hours post injection on subjects treated with different samples prepared in accordance with various aspects of the present disclosure;

FIG. 7F is a graphical demonstration comparing ALT activity at 60 hours post injection on subjects treated with different samples prepared in accordance with various aspects of the present disclosure; and

FIG. 7G is a graphical demonstration comparing cTnl concentrations at 60 hours post injection on subjects treated with different samples prepared in accordance with various aspects of the present disclosure.

Corresponding reference numerals indicate corresponding parts throughout the several views of the drawings.

### DETAILED DESCRIPTION

Example embodiments will now be described more fully with reference to the accompanying drawings.

Example embodiments are provided so that this disclosure will be thorough and will fully convey the scope to those who are skilled in the art. Numerous specific details are set forth such as examples of specific components, devices, and methods, to provide a thorough understanding of embodiments of the present disclosure. It will be apparent to those skilled in the art that specific details need not be employed, that example embodiments may be embodied in many different forms and that neither should be construed to limit the scope of the disclosure. In some example embodiments, well-known processes, well-known device structures, and well-known technologies are not described in detail.

The terminology used herein is for the purpose of describing particular example embodiments only and is not intended to be limiting. As used herein, the singular forms “a,” “an,” and “the” may be intended to include the plural forms as well, unless the context clearly indicates otherwise. The terms “comprises,” “comprising,” “including,” and “having” are inclusive and therefore specify the presence of stated features, integers, steps, operations, elements, and/or components, but do not preclude the presence or addition of one or more other features, integers, steps, operations, elements, components, and/or groups thereof. The method steps, processes, and operations described herein are not to be construed as necessarily requiring their performance in the particular order discussed or illustrated, unless specifically identified as an order of performance. It is also to be understood that additional or alternative steps may be employed.

When an element or layer is referred to as being “on,” “engaged to,” “connected to,” or “coupled to” another element or layer, it may be directly on, engaged, connected, or coupled to the other element or layer, or intervening elements or layers may be present. In contrast, when an element is referred to as being “directly on,” “directly engaged to,” “directly connected to,” or “directly coupled to” another element or layer, there may be no intervening elements or layers present. Other words used to describe the relationship between elements should be interpreted in a like fashion (e.g., “between” versus “directly between,” “adjacent” versus “directly adjacent,” etc.). As used herein, the term “and/or” includes any and all combinations of one or more of the associated listed items.

Although the terms first, second, third, etc. may be used herein to describe various elements, components, regions, layers, and/or sections, these elements, components, regions, layers, and/or sections should not be limited by these terms. These terms may be only used to distinguish one element, component, region, layer or section from another region, layer, or section. Terms such as “first,” “second,” and other numerical terms when used herein do not imply a sequence or order unless clearly indicated by the context. Thus, a first element, component, region, layer, or section discussed below could be termed a second element, component, region, layer, or section without departing from the teachings of the example embodiments.

Spatially relative terms, such as “inner,” “outer,” “beneath,” “below,” “lower,” “above,” “upper,” and the like, may be used herein for ease of description to describe one element or feature's relationship to another element(s) or feature(s) as illustrated in the figures. Spatially relative terms may be intended to encompass different orientations of the device in use or operation in addition to the orientation depicted in the figures. For example, if the device in the figures is turned over, elements described as “below” or “beneath” other elements or features would then be oriented “above” the other elements or features. Thus, the example term “below” can encompass both an orientation of above and below. The device may be otherwise oriented (rotated 90 degrees or at other orientations) and the spatially relative descriptors used herein interpreted accordingly.

Example embodiments will now be described more fully with reference to the accompanying drawings.

The present disclosure provides methods that allow physicians and/or prescribers and/or administrators to track drug doses anywhere in a subject and to maintain drug concentrations within therapeutic windows by applying or removing (turning ON/OFF) an external stimulus (e.g., near infrared laser (NIR)) to minimize side effects while increasing therapeutic effects. The physicians and/or prescribers and/or administrators may administer a nanocomposite that includes a biodegradable polymer shell that at least partially encompasses a core including a collection or cluster or agglomeration of magnetic nanoparticles. In at least one example embodiment, the biodegradable polymer shell may include a polymeric material selected from the group consisting of: poly(lactide-co-glycolide acid) (PLGA), polymerized ursodeoxycholic acid (PUDCA), polyethylenimine (PEI), chitosan, poly(d-lactic acid), poly(s-caprolactone) (PCL), polylactic acid, polyanhydrides, polyphosphoesters, polyurethanes, poly(caprolactone), hyaluronic acid, albumin, polysaccharides, and combinations thereof). The magnetic nanoparticles must be monodispersable. In at least one example embodiment, the magnetic nanoparticles may have an average particle size less than about 100 nanometers (nm). In at least one example embodiment, the magnetic nanoparticles may include Fe₃O₄ nanoparticles. The shell and/or core may be loaded with one or more drugs, including, for example, chemotherapy drugs, such as doxorubicin (DOX). In at least one example embodiment, the nanocomposite may be as described in U.S. Pub. No. 2021/0315476, titled DEPTH-INDEPENDENT METHOD FOR IN-VIVO DRUG RELEASE MONITORING AND QUANTIFICATION BASED ON MAGNETIC PARTICLE IMAGING and listing Bryan R. Smith, Xingjun Zhu, and Jianfeng Li as inventors, the entire disclosure of which is incorporated herein by reference.

The physicians and/or prescribers and/or administrators may monitor and quantify drug release in vitro and/or in vivo using, for example, magnetic particle imaging (“MPI”). For example, released doxorubicin and MPI signal have been shown to display a linear correlation (R²=0.99). MPI signal intensity increases at least in part due to increased Brownian relaxation of the magnetic nanoparticles (e.g., Fe₃O₄ nanoparticles), which may become larger (e.g., greater than or equal to about 1 to less than or equal to about 3 magnetic cores) as the biodegradable polymer shell (including, for example, PLGA) degrades upon the irradiation of external stimulus (e.g., near infrared laser (NIR)). Because the magnetic nanoparticles are rotated and/or released concurrently with the one or more drugs, drug release can be monitored using a linear correlation between the drug release and MPI signal.

Using the collected information, the physicians and/or prescribers and/or administrators may elect to initiated and/or accelerate degradation of the administered nanocomposite using, for example, optical stimulus, such as generated using near-infrared (NIR) light, to maintain drug release (from the nanocomposite) within therapeutic windows. The physicians and/or prescribers and/or administrators may monitor drug release, for example, using MPI, both before and after stimulation, to continue to adjust or tune degradation as appropriate for targeted treatments or therapies. By targeting release using the quantifying and tuning methods, in addition to improved efficacy, off-target toxicity can also be reduced, including for example, reducing and/or limiting toxicity to normal organs and not increasing cardiotoxicity or hepatotoxicity.

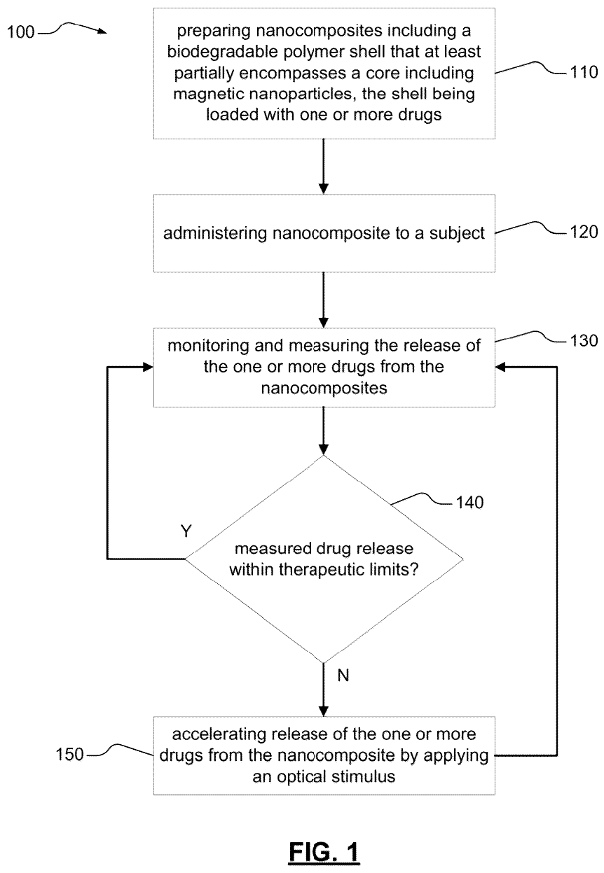

An example method 100 for targeting and tuning drug release or application in vitro and/or in vivo is illustrated in FIG. 1. As illustrated, the method 100 may include monitoring and/or collecting gathering information 130 in vitro and/or in vivo regarding the release of one or more drugs from one or more nanocomposites, each including a biodegradable polymer shell that at least partially encompasses a core including collection or cluster or agglomeration of magnetic nanoparticles, where the shell and/or core are loaded with one or more drugs. The biodegradable polymer shell may substantially surround greater than or equal to about 50%, optionally greater than or equal to about 60%, optionally greater than or equal to about 70%, optionally greater than or equal to about 80%, optionally greater than or equal to about 90%, optionally greater than or equal to about 91%, optionally greater than or equal to about 92%, optionally greater than or equal to about 93%, optionally greater than or equal to about 94%, optionally greater than or equal to about 95%, optionally greater than or equal to about 96%, optionally greater than or equal to about 97%, optionally greater than or equal to about 98%, optionally greater than or equal to about 99%, optionally greater than or equal to about 99.5%, optionally greater than or equal to about 99.6%, optionally greater than or equal to about 99.7%, optionally greater than or equal to about 99.8%, or optionally greater than or equal to about 99.9%, of a total surface of the core.

In at least one example embodiment, the monitoring 130 may include magnetic particle imaging (“MPI”), where the MPI detects superparamagnetic nanoparticle tracers (e.g., Fe₃O₄ nanoparticles) using a MPI scanner. The MPI scanner is configured to acquire a three-dimensional image of the tracer distribution. As shown in the below examples, the intensity of the MPI signals may vary in response to the release of the one or more drugs from the nanocomposites. In at least one example embodiment, a linear correlation may be demonstrated between the MPI signals and release of the one or more drugs from the nanocomposites.

The method 100 further includes, concurrently or subsequently to the monitoring 130, analyzing 140 the collected drug release information to determine if the released drug(s) are within the assigned therapeutic window. If the collected drug release information indicates that the release of the one or more drugs from the nanocomposite is within therapeutic levels, the method 100 will continue (passively or in response to active steps by the physician and/or prescriber and/or administrator or controller and/or processor in communication with the same) to monitor 130 in vitro and/or in vivo the release of one or more drugs from nanocomposites. If however, the collected drug release information indicates that the release of the one or more drugs from the nanocomposite is outside of therapeutic levels, namely below therapeutic levels, the method 100 may include accelerating 150 release of the one or more drugs from the nanocomposite by applying (passively or in response to active steps by the physician and/or prescriber and/or administrator or controller and/or processor in communication with the same) precision-controlled optical stimulus to a selected region, including, for example, the tumor of interest. The optical stimulus is precision-controlled in that its application (in space) can be exactly controlled and also application time, irradiation power, and/or wavelength of the optical stimulus can be precisely controlled. In at least one example embodiment, the optical stimulus may include near-infrared (NIR) light irradiation. As shown in the below examples, in response to the optical stimulus, and in particular, the near-infrared light irradiation, the biodegradable polymer shell degradation to release and disassemble the magnetic nanoparticles and the one or more drugs.

The optical stimulus may be applied to the target region for a selected period. For example, the optical stimulus may be applied for a period greater than or equal to about 1 minute to less than or equal to about 20 minutes, and in certain aspects, optionally greater than or equal to about 1 minute to less than or equal to about 7 minutes. The selected period is tunable in response to the collected drug release information (i.e., the real-time data). For example, the length of the period may be selected to be smaller when the collected drug release information indicates that the actual release of the one or more drugs is nearer to the therapeutic level(s), and the length of the period may be selected to be larger when the collected drug release information indicates that the actual release of the one or more drugs is further from the therapeutic level(s). Similarly, an intensity of the optical stimulus may be varied in response to the collected drug release information. In at least one example embodiment, the intensity may range from greater than or equal to about 0.5 W/cm² to less than or equal to about 10 W/cm². For example, when the measured concentration in the tumor is near the therapeutic window, the intensity may be about 0.5 W/cm², and when the measured concentration in the tumor is significantly less than the therapeutic window, the intensity may be greater than or equal to about 2 W/cm² to less than or equal to about 10 W/cm².

After the application of the optical stimulus, the method 100 may return to monitoring 130 in vitro and/or in vivo the release of one or more drugs from nanocomposites. Method steps 130-140 may continue until the nanocomposites have been fully disabled and entire concentrations of the one or more drugs released.

In at least one example embodiment, the method 100 may include preparing 110 the nanocomposites. The nanocomposites may be prepared as detailed in U.S. Pub. No. 2021/0315476, titled DEPTH-INDEPENDENT METHOD FOR IN-VIVO DRUG RELEASE MONITORING AND QUANTIFICATION BASED ON MAGNETIC PARTICLE IMAGING and listing Bryan R. Smith, Xingjun Zhu, and Jianfeng Li as inventors, the entire disclosure of which is incorporated herein by reference. For example, preparing 110 the nanocomposites may include contacting superparamagnetic nanoclusters (“SPNC”) and the one or more drugs to form drug-loaded superparamagnetic nanocomposites (“SPNCD”). In at least one example embodiment, contacting the nanoclusters and the one or more drugs may include adding the nanoclusters and magnetic nanoparticles, consecutively or concurrently, to an aqueous solution (e.g., deionized water) and applying a mixing force (for example, using a magnetic stirrer) for a selected time period. In at least one example embodiment, the method 100 may also include preparing the nanoclusters. Preparing the nanoclusters may include, for example, contacting the magnetic nanoparticles and the biodegradable polymer. In at least one example embodiment, contacting the magnetic nanoparticles and the biodegradable polymer may include adding the magnetic nanoparticles and the biodegradable polymer consecutively or concurrently, to a solvent (e.g., polyvinyl alcohol) and applying a mixing force (for example, using a magnetic stirrer) for a selected time period, and then contacting the formed mixture to a sonicator to form emulsions.

In at least one example embodiment, the method 100 may administering by a physician and/or prescriber and/or administrator nanocomposites to a human or non-human subject. Administering the nanocomposites may include intratumoral injection.

Certain features of the current technology are further illustrated in the following non-limiting examples.

### Example 1

### Preparation and Characterization of SPNCD

Example SPNCD may be prepared in accordance with various aspects of the present disclosure. For example, Fe₃O₄ nanoparticles may be mixed with PLGA in a solvent (e.g., chloroform) to form a mixture. In at least one example embodiment, about 1 milligram of the Fe₃O₄ nanoparticles may be mixed with about 5 milligrams of the poly(lactide-co-glycolide acid). In at least one example embodiment, the Fe₃O₄ nanoparticles may have an average particle diameter of about 25 nanometers (nm) and a molecular weight of about 1 milligram (mg). In at least one example embodiment, the solvent may include chloroform.

The mixture may be contacted with a first aqueous solution to form an emulsified solution. For example, in at least one example embodiment, the mixture may be added to about 4 millimeters (mL) of the first aqueous solution to form the emulsified solution. In at least one example embodiment, the first aqueous solution may be a first polyvinyl alcohol (PVA) aqueous solution. In at least one example embodiment, the first polyvinyl alcohol aqueous solution may be a 3% W/V polyvinyl alcohol aqueous solution. In at least one example embodiment, vortex and/or sonication forces may be applied to the emulsified solution.

The emulsified solution may be contacted with a second aqueous solution to form an emulsion. For example, in at least one example embodiment, the emulsified solution may be injected dropwise into the second aqueous solution. In at least one example embodiment, the emulsified solution may be contacted with about 20 milliliters of the second aqueous solution. In at least one example embodiment, the second aqueous solution may be a polyvinyl alcohol (PVA) aqueous solution. In at least one example embodiment, the second polyvinyl alcohol aqueous solution may be a 1% W/V polyvinyl alcohol aqueous solution.

The emulsion may be stirred for a first preselected period to evaporate the solvent and form the nanoclusters. In at least one example embodiment, the first preselected period may be greater than or equal to about 8 hours to less than or equal to about 12 hours.

The nanoclusters may be washed one or more times, for example, with deionized water, and then dispersed in deionized water (pH=7.4). In at least one example embodiment, the nanoclusters may be washed three times.

Doxorubicin (DOX)-loaded nanoclusters may be prepared by contacting DOX to the deionized water including the magnetic nanoparticles. In at least one example embodiment, the deionized water including the nanoparticles and DOX may be mixed for a second preselected period. In at least one example embodiment, the second preselected period may be greater than or equal to about 8 hours to less than or equal to about 12 hours.

The resulting SPNCD may be separated from the deionized water including any residual nanoparticles and/or DOX using, for example, centrifugation. In at least one example embodiment, the centrifugation may have a force of about 6,000×g. The resulting SPNCD may be washed one or more times, for example, with deionized water, and then dispersed in deionized water (pH=7.4). In at least one example embodiment, the nanoclusters may be washed three times.

A Nanodrop may be used to confirm the successfully loading of DOX on the SPNC. A Malvern Zetasizer may be used to characterize the diameter and ζ-potential of the SPNC and the SPNCD. Transmission electron microscopy (“TEM”) (e.g., JEOL 2200FS, TOYKO) and scanning electron microscopy (“SEM”) (e.g., JEOL 7500F, TOYKO) may be used to characterize the morphology of SPNC and SPNCD.

### Example 2

### Preparation and Characterization of SPNC and SPNCD

Example SPNC and SPNCD may be prepared in accordance with various aspects of the present disclosure. For example, in at least one example embodiment, SPNC including an Fe₃O₄ core and PLGA shell may be prepared via a co-precipitation method. DOX may be loaded into the SPNC to form the SPNCD.

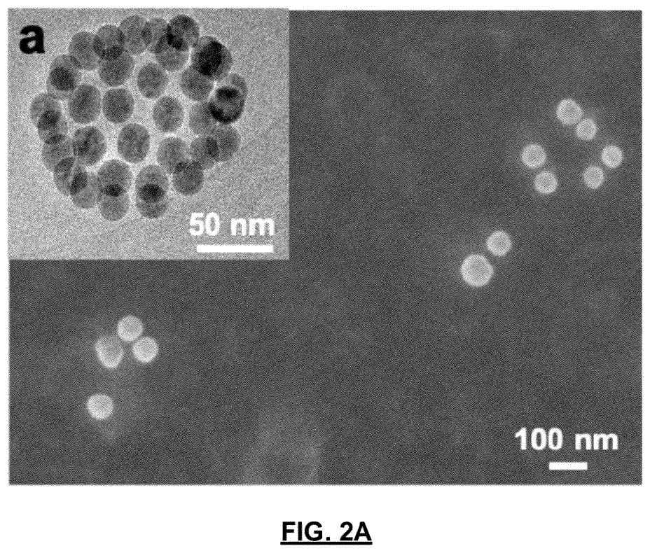

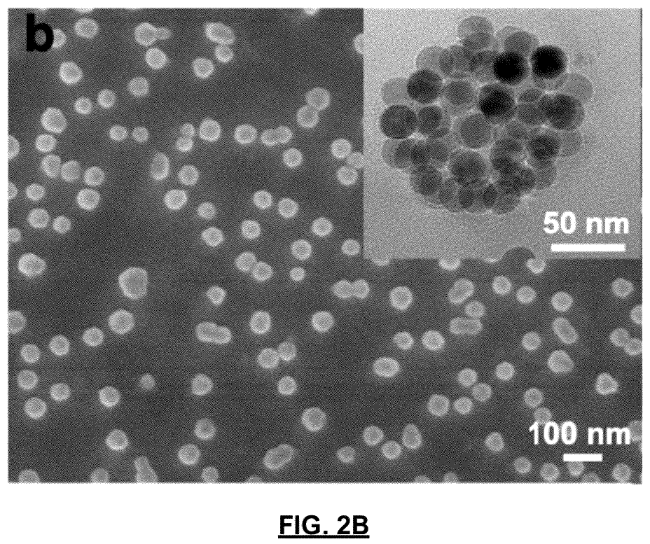

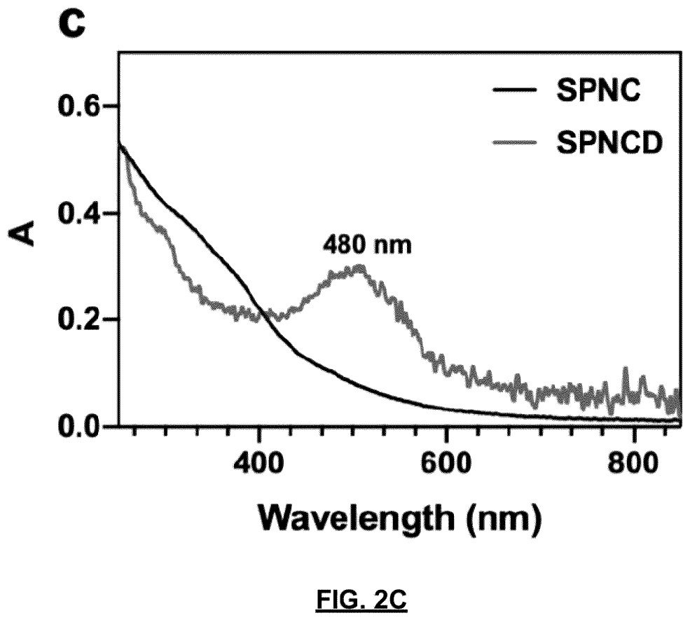

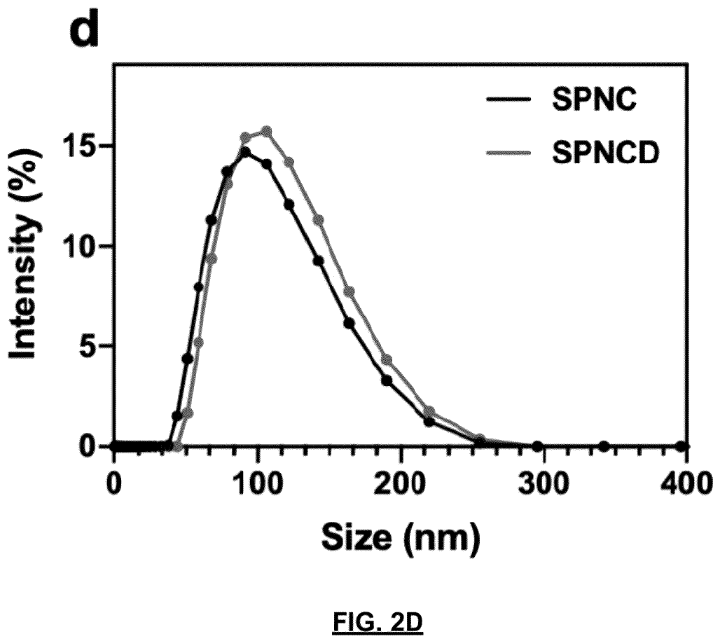

FIG. 2A is a SEM image of the as-prepared SPNC with a TEM insert of the as-prepared SPNC, where the scale of the SEM is 100 nanometers, and the scale of the TEM is 50 nanometers. FIG. 2B is a SEM image of SPNCD with a TEM insert of SPNCD, where the scale of the SEM is 100 nanometers, and the scale of the TEM is 50 nanometers. FIG. 2C is a UV-Vis adsorption spectra of SPNC and SPNCD, where the x-axis represents wavelength in nanometers and the y-axis represents absorbance (“A”). FIG. 2D is a dynamic light scatting (“DLS”) of SPNC and SPNCD, where the x-axis represents size in nanometers and the y-axis represents intensity in percent.

The SEM, TEM, and DLS of the as-prepared SPNC shows that the nanocomposites display a nearly spherical morphology with an average diameter of about 91 nanometers. Further, as illustrated, after the DOX was loaded into the SPNC to form the SPNCD, the spherical morphology of the nanocomposites remains. The SPNCD may have an average diameter of about 106 nanometers.

The UV-Vis adsorption spectroscopy proves successful DOX loading in SPNC based on the emergence of a characteristic new peak at 480 nanometers in the SPNC spectrum. Zeta potential measurements of SPNC and SPNCD may, additionally or alternatively, be used to corroborated the successful loading process of positively charged DOX based on the shift of SPNC charge from −17.8 mV to −9.26 mV.

### Example 3

### Drug Release Monitoring

SPNCD phosphate buffered saline (“PBS”) solutions (including, for example, a first SPNCD PBS solution having a pH of 7.4 and a second SPNCD PBS solution having a pH of 6.5) and SPNCD 10% serum solutions (including, for example, a first SPNCD 10% serum solution having a pH of 7.4 and a second SPNCD 10% serum solution having a pH of 6.5) may be imaged at one or more timepoints using MPI (e.g., Magnetic Insight, USA). In at least one example embodiment, the SPNCD 10% serum solutions may be prepared by adding 10 mL serum to 100 mL of PBS having a pH of 7.4 and 6.5, respectively. In at least one example embodiment, the SPNCD PBS solutions and SPNCD 10% serum solutions may each be imaged six times, including, for example, at 0, 1, 2, 5, 24, 48 hours, using MPI.

MPI signal intensity may be quantified by choosing regions-of-interest using, for example, VivoQuant software. A plate reader (e.g., SpectraMax M3, Molecular Device) may be used to measure the fluorescent excitation of DOX released in the solution at 488 nanometers after each MPI scan.

To study optically triggered drug release using near-infrared (NIR) (e.g., CivilLaser, China), the SPNCD PBS solution may be irradiated for one or more periods and followed by MPI scan and plate reader testing. In at least one example embodiment, the NIR may have a wavelength of about 808 nanometers, an intensity about 1 W/cm², and a spot size of about 4.2 millimeters by 2.6 millimeters. In at least one example embodiment, the one or more periods may include a first period, a second period, a third period, and a fourth period. In at least one example embodiment, the first period may be about 1 minute. In at least one example embodiment, the second period may be about 3 minutes. In at least one example embodiment, the third period may be about 5 minutes. In at least one example embodiment, the fourth period may be about 7 minutes.

### Example 4

### Drug Release Monitoring

In at least one example embodiment, a first portion or group of SPNCD nanocomposites may be dispersed in pH=7.4 PBS to simulate blood pH; a second portion or group of SPNCD nanocomposites may be dispersed in pH=6.5 PBS to simulate tumor acidic environment; a third portion or group 10% serum at pH 6.5 to simulate blood plasma; and a fourth portion or group 4T1 cells demonstrating drug release in cell line.

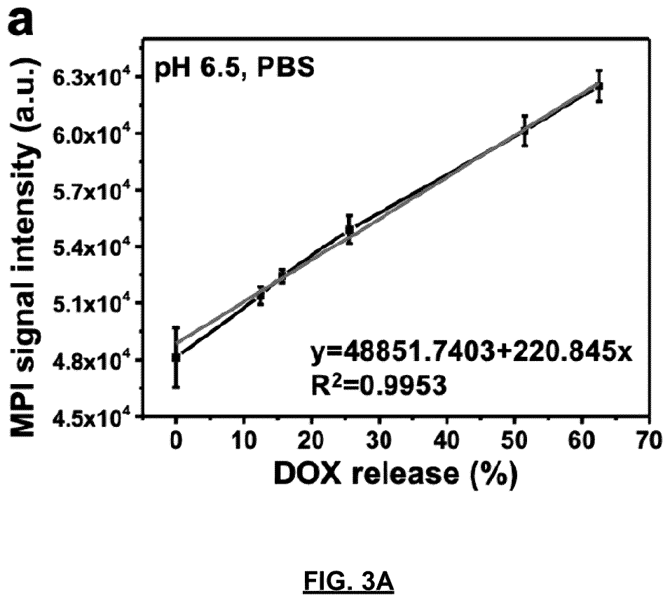

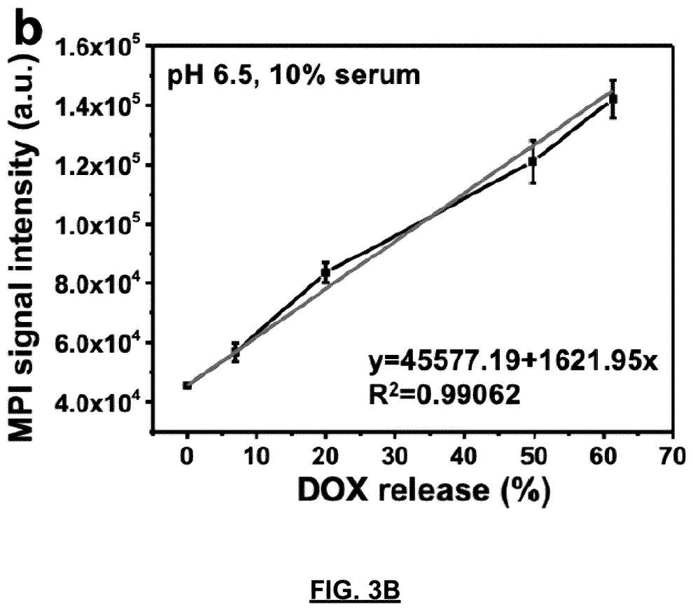

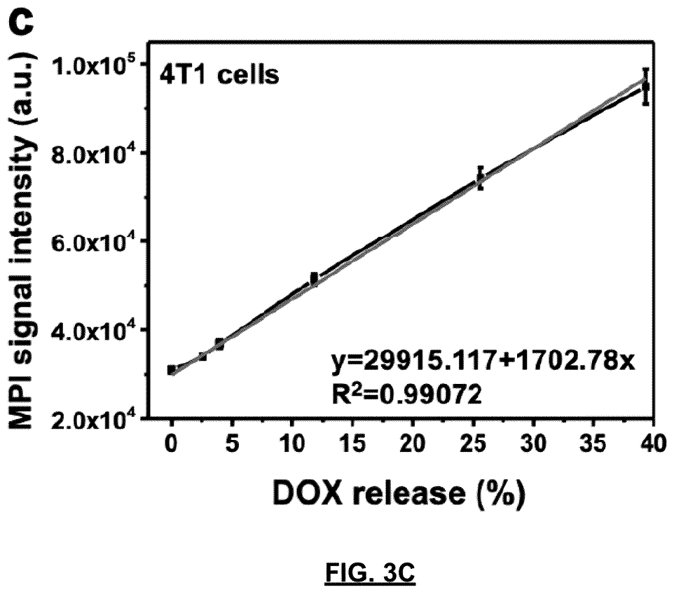

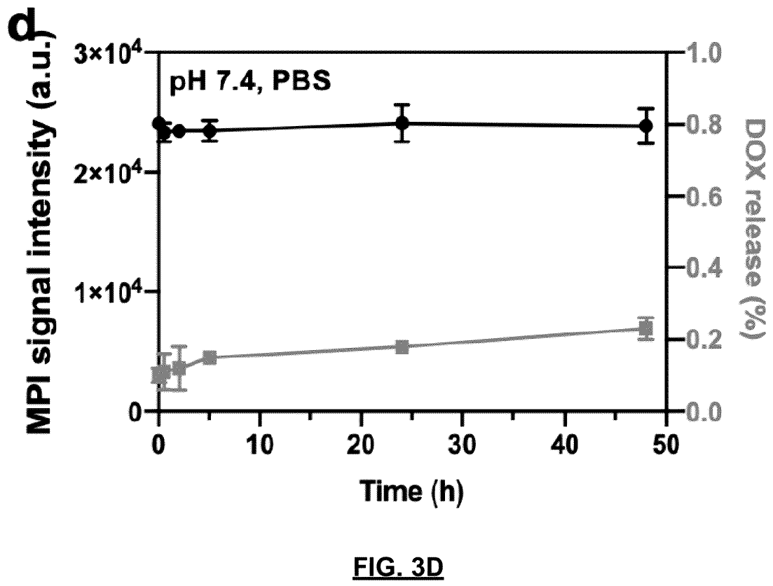

DOX release may be monitored via quantification of MPI signal changes arising from SPNCD degradation due to acidic pH without external energy. For example, FIG. 3A graphical demonstration illustrating a relationship between DOX release and MPI signal intensity for the second group, where the x-axis represents DOX release in percent (%) and the y-axis represents MPI signal intensity in arbitrary units (a.u.); FIG. 3B is a graphical demonstration illustrating a relationship between DOX release and MPI signal intensity for the third group, where the x-axis represents DOX release in percent (%) and the y-axis represents MPI signal intensity in arbitrary units (a.u.); FIG. 3C is a graphical demonstration illustrating a relationship between DOX release and MPI signal intensity for the fourth group, where the x-axis represents DOX release in percent (%) and the y-axis represents MPI signal intensity in arbitrary units (a.u.); and FIG. 3D is a graphical demonstration illustrating a relationship between DOX release and MPI signal intensity for the first group, where the x-axis represents DOX release in percent (%) and the y-axis represents MPI signal intensity in arbitrary units (a.u.).

As illustrated, DOX release and MPI signal increased monotonically as time increased. For example, about 61.3%, about 62.4% and about 40% DOX was released at for each of the second, third, and fourth groups after 48 hours. The SPNCD MPI signal change linearly corresponded with the DOX release rate across the different time points of the release process in all incubation conditions, with an early rapid increase followed by a slower increase. A linear correlation between MPI signal and DOX release was obtained with R²=0.9953 for the second group, as illustrated in FIG. 3A; with R²=0.9906 for the third group, as illustrated in FIG. 3B; and with R²=0.9907 for the fourth group, as illustrated in FIG. 3C. Importantly, as illustrated in FIG. 3D, less than 2% DOX was released at neutral pH of 7.4 in PBS buffer and pH 7.4 in serum during the tested time period (e.g., 48 hours), indicating that SPNCD nanocomposites feature pH-responsive release and are stable at a pH mimicking neutral extracellular environments and blood.

### Example 5

Drug Release Monitoring in 4T1 Cells with/without NIR

Energy can be applied to control drug release from SPNCD. Optical (NIR)-based energy allows fine control over energy intensity, time, and precise localization.

Mouse mammary carcinoma 4T1 cells were used to study intracellular SPNCD drug release. For example, 4T1 cells were deposited into black breakable 96-well plates and cultured at 37° C. and 5% CO₂ for 24 hours. The cells were then incubated with SPNCD at a concentration of 90 micrograms (μg)/millimeters at different time periods (e.g., 0, 0.5, 2, 5, 24, 48 hours). The fluorescence excitation of DOX in the cell samples was measured at 488 nanometers with a plate reader (e.g., SpectraMax M3, Molecular Device) to determine the quantity of DOX released from SPNCD and MPI was used to quantify the MPI signal in samples.

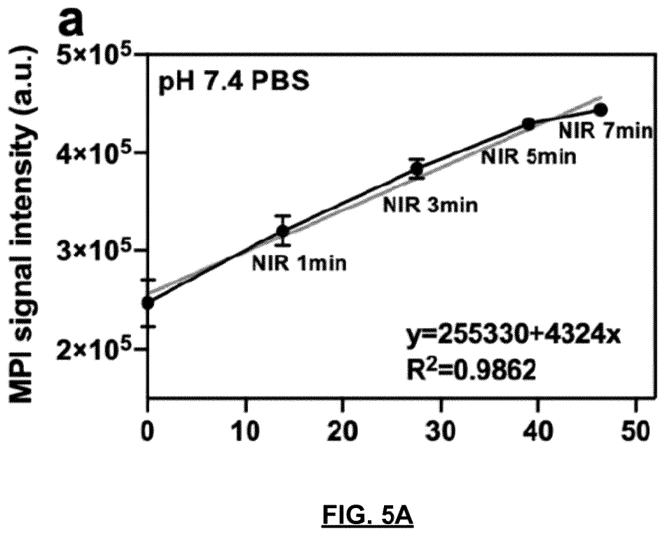

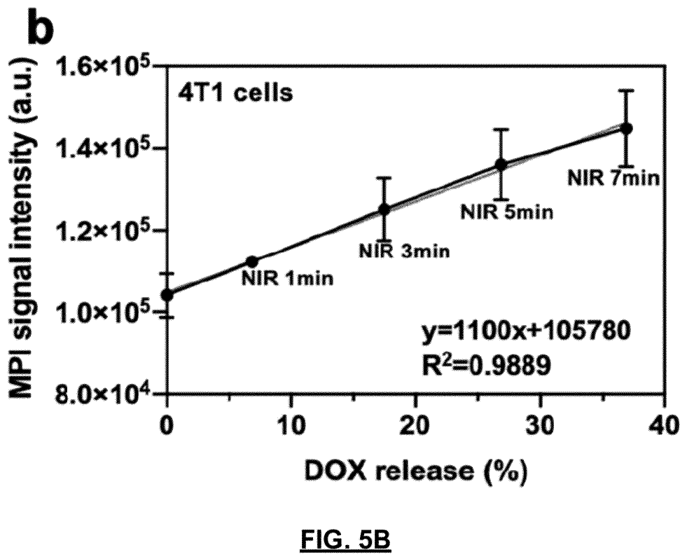

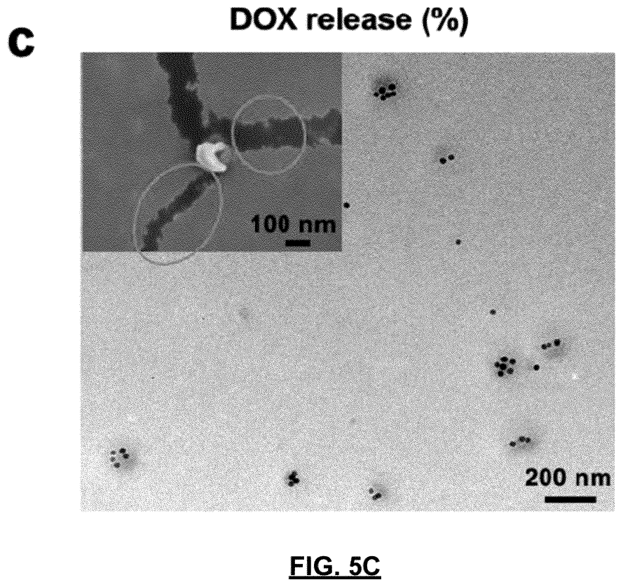

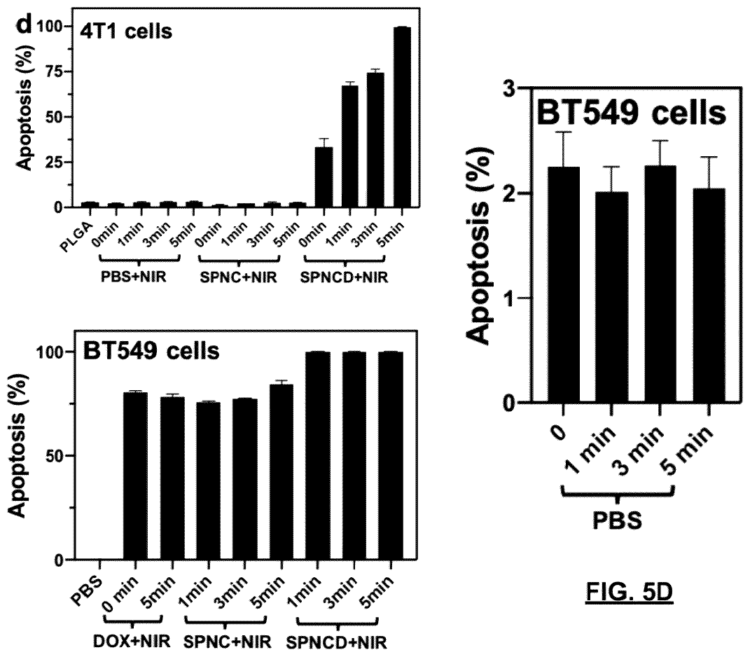

To quantify and the drug release triggered by NIR in 4T1 cells, 4T1 cells were deposited into black breakable 96-well plates and cultured at 37° C. and 5% CO₂ for 24 hours. The cells were then incubated with SPNCD at a concentration of 90 micrograms/milliliter and irradiated with NIR for 1, 3, 5 and 7 minutes, followed by MPI scan and plate reader testing. For example, FIG. 5A is a graphical demonstration illustrating a relationship between Doxorubicin release and magnetic particle imaging signal intensity for SPNCD dispersed in pH=7.4 PBS to simulate blood pH, when NIR-stimulated drug release occurs at 1 minute, 3 minutes, 5 minutes and 7 minutes, where the x-axis represents DOX release in percent and the y-axis represents MPI signal intensity in arbitrary units (a.u.); and FIG. 5B is a graphical demonstration illustrating a relationship between Doxorubicin release and magnetic particle imaging signal intensity for 4T1 cells, when NIR-stimulated drug release occurs at 1 minute, 3 minutes, 5 minutes and 7 minutes, where the x-axis represents DOX release in percent and the y-axis represents MPI signal intensity in arbitrary units (a.u.). As illustrated in FIG. 5A, DOX release was increased by 47% after 7 minutes of NIR (1W, 808 nm) irradiation. As illustrated in FIG. 5B, DOX release was increased by 36.9% after 7 minutes of NIR (1W, 808 nm) irradiation.

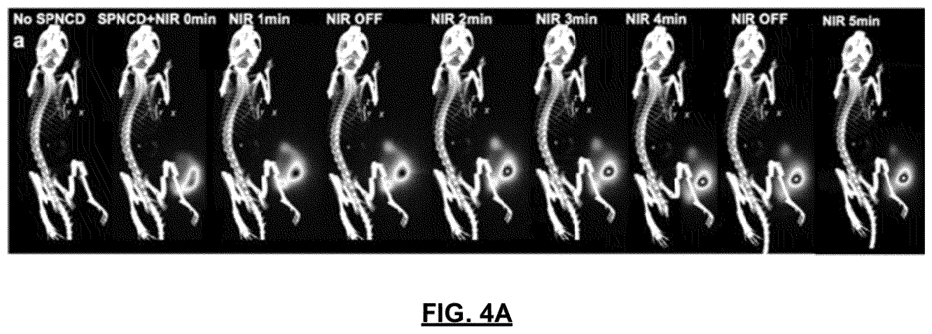

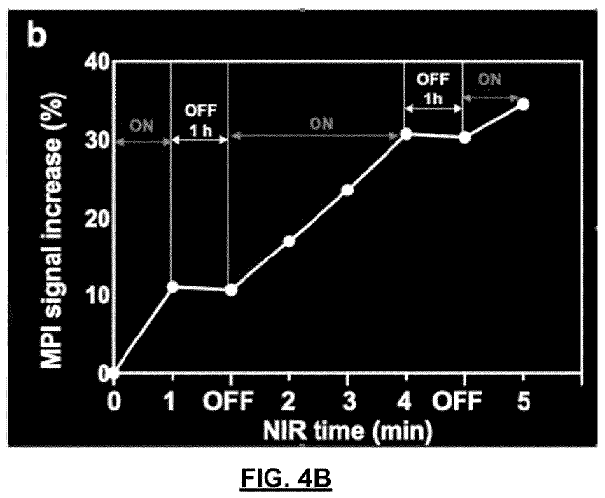

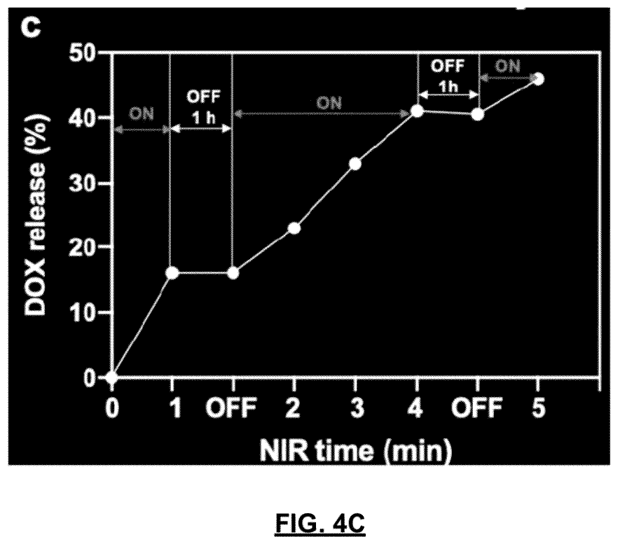

To explore the potential causes underlying the MPI signal change during drug release using NIR, the SPNCD nanocomposites may be imaged by SEM and TEM, for example, as illustrated in FIG. 5C. As illustrated, SPNCD nanocomposites clearly undergo degradation upon 7 min NIR irradiation. Compared with the morphology of fresh or untreated SPNCD as illustrated in FIG. 2B, the spherical morphology of NIR-irradiated SPNCD illustrated in FIG. 4C appears damaged. The SPNCD average size as measured using FIG. 4C, decreased (e.g., from about 106 nanometers to about 65 nanometers) and the average number of iron oxide nanoparticles per SPNCD was reduced (e.g., from about 30 to about 4) after 7 minutes of NIR irradiation, which indicates that iron oxide nanoparticles were released from SPNCD. The free iron oxide nanoparticles are circled on the SEM.

### Example 6

### Apoptosis Assay

Cell apoptosis of the 4T1 cells, for example prepared as detailed in Example 3, may be evaluated by assessing Annexin V-FITC/7AAD using, for example, flow cytometry. For example, the 4T1 cells may be deposited into 12-well cell culture plates (5×10⁴ cells/well) and cultured at 37° C. and 5% CO₂ for 24 hours. The cells may then be treated with PLGA, PBS+NIR (0, 1, 3 and 5 minutes), SPNC+NIR (90 μg/mL, 0, 1, 3 and 5 minutes), and/or SPNCD+NIR (90 μg/mL, 0, 1, 3 and 5 minutes) and further cultured at 37° C. and 5% CO₂ for another 24 hours. The cells may then be stained with Annexin V-FITC/7AAD (e.g., Biolegend, USA) and analyzed using, for example, flow cytometry.

The biological effects of NIR+SPNCD on 4T1 cells and BT549 cells may be explored using flow cytometric apoptosis assay. For example, FIG. 5A includes graphical demonstrations illustrating flow cytometric apoptosis assay for different samples as for different cell types, where the x-axis represents time and the y-axis represents apoptosis in percent. The data reveal that PLGA NPs and SPNC NPs alone display low toxicity to 4T1 cells (less than ~2.9% apoptotic cells), the mild heat generated by NIR alone (PBS-NIR group and SPNC-NIR group) has no significant killing effects on cells either (less than about 3.2% apoptotic cells). However, when SPNCD+NIR groups were NIR-irradiated, the apoptosis percentage increased incrementally with NIR irradiation time to nearly 100% due to increased DOX release, resulting in increased cell apoptosis. The SPNCD+0 min NIR group showed about 34% apoptosis due to the acidic intracellular pH, resulting in DOX release. Comparing the apoptosis rate between BT549 cells and 4T1 cells, it is clear that SPNC-NIR and SPNCD-NIR induced more cell apoptosis on BT549 cells than 4T1 cells, which is likely because the nanoparticle concentration used for BT549 cells is 10 times higher than that for 4T1 cells, so as to lead to higher PTT effect. Interestingly, there is no apoptosis in the instance of the PBS-NIR group, which further indicates NIR alone will not induce cell apoptosis.

### Example 7

### Three-Dimensional Cell Viability Assay

Three-dimensional cell viability experiments may be conducted to identify appropriate DOX doses for in vivo experiment and quantify the half maximal inhibitory concentration (“IC50”).

For example, at greater than or equal to about 80% to less than or equal to about 90% confluence, 4T1 and BT549 cells may be dispersed using 0.05% Trypsin-EDTA (e.g., Invitrogen) then re-suspended in cell culture media, where the 4T1 are DOX sensitive cells and BT549 are DOX resistant cells.

The cells may be plated into an ultra-low adherent plates with black opaque sides and then centrifuged. In at least one example embodiment, the ultra-low adherent plates may include 96 wells. In at least one example embodiment, about 100 microliters may be disposed in each well. In at least one example embodiment, about 5×10⁴ cells may be disposed in each well. In at least one example embodiment, the plates may be centrifuged for about 1 minute at about 120×g.

The cells may be dosed within four hours of plating with DOX. In at least one example embodiment, at least a first portion of wells may receive a first amount of DOX. The first amount may be about 0.014 micromolar (μM). In at least one example embodiment, at least a second portion of the wells may receive a second amount of DOX. The second amount may be about 0.08 micromolar. In at least one example embodiment, at least a third portion of the wells may receive a third amount of DOX. The third amount may be about 0.4 micromolar. In at least one example embodiment, at least a fourth portion of the wells may receive fourth amount of DOX. The fourth amount may be about 2 micromolars. In at least one example embodiment, at least a fifth portion of the wells may receive a fifth amount of DOX. The fifth amount may be about 10 micromolars.

After a desired DOX incubation period, a cell viability reagent (e.g., cell-titer 3D ATP-glo) may be added to the plate following the manufacturer's instructions. In at least one example embodiment, the DOX incubation period may be about 120 hours at about 37° C. and about 5% CO₂.

Luminescence of the viability reagent in each well may then be measured using, for example, a plate reader (e.g., SpectraMax M3, Molecular Device) and cell viability may be measured as percent viability with respect to DOX concentration. Dose-response growth curves may be generated from measurements of triplicate wells. Data fitting may be performed using, for example, Graphpad Prism 7.

### Example 8

### Tumor Xenografts

Triple-negative breast cancer cell line (luciferase-expressing 4T1 cells and BT549 cells) and BT549 cells were collected, centrifuged, and resuspended in PBS.

The 4T1 cells may be implanted into the mammary fat pads of six-week-old female Balb/c mice. In at least one example embodiment, about 1×10⁶ cells may be implanted per mouse.

The BT549 cells may be implanted into the mammary fat pads of six-week-old female nude mice. In at least one example embodiment, about 1×10⁷ cells may be implanted per mouse.

Orthotopic tumor-bearing mice were considered ready for in vivo studies when the tumor volume reached or exceeded 100 mm³. The tumor volume may be calculated, for example, using the following equation:

$$V = {W}^{2} \times L / 2$$

where W and L are width and length of the tumor measured by a caliper, respectively.

### Example 9

### Drug Release Monitoring In Vivo

In at least one example embodiment, SPNCD may be injected intratumorally into an orthotopically-implanted Balb/c 4T1 murine breast cancer model. MPI may be performed before and after SPNCD injection and after each NIR irradiation in order to quantify changes in signal intensity. The 4T1 tumor-bearing Balb/c mice may be MPI imaged prior to intratumoral injection of SPNCD to provide a baseline measure of MPI signal (typically 0) in the mice. In at least one example embodiment, the mice may then be intratumorally injected with SPNCD at a concentration of 90 μg/mL in 50 μL of PBS.

The mice may then be irradiated with the NIR laser (e.g., 808 nm, 1W) for greater than or equal to about 1 minute to less than or equal to about 5 minutes. The mice may be imaged using, for example, Micro-CT (e.g., Quantum GX2, PerkinElmer) immediately after each MPI time point to provide overlaid anatomic information for MPI by superimposing CT images with MPI images, using, for example, VivoQuant software. Three fiducials markers (e.g., VivoTrax, Magnetic Insight) may be used to register the overlay of MPI and CT images. For example, FIG. 4A provides merged MPI/CT images of 4T1 tumor-bearing mice injected intratumorally with SPNCD and stimulated with NIR for periods between greater than or equal to 1 minute to less than or equal to about 5 minutes, with two one-hour periods, after 1 minute and 4 minutes with no NIR stimulation. FIG. 4B is a graphical demonstration illustrating a relationship between the time of NIR stimulations and MPI signal increases, where the x-axis represents NIR time in minutes and the y-axis represents MPI signal increases in percent. FIG. 4C is a graphical demonstration illustrating a relationship between time of NIR stimulations and DOX release, where the x-axis represents NIR time in minutes and the y-axis represents DOX release in percent.

As illustrated, the signal intensity increased by about 11% after 1 minute of NIR irradiation and continued to increase with increasing NIR irradiation to about 34.5% after 5 minutes, and about 46% of DOX molecules in the SPNCD nanocomposite were release within the tumor after using NIR for 5 minutes, suggesting that MPI signal intensity can be controlled simply by turning NIR on or off. The MPI signal displayed essentially no change when NIR was off for the selected breaks after 1 minute and 4 minutes.

### Example 10

### Short-Term Tumor Inhibition

The 4T1 tumor-bearing Balb/c mice may be randomly divided into six groups. In at least one example embodiment, each group may include three mice. In at least one example embodiment, a first group may receive PLGA. In at least one example embodiment, a second group may receive SPNCD. In at least one example embodiment, a third group may receive SPNCD and may be irradiated with NIR for 1 minute. In at least one example embodiment, a fourth group may receive SPNCD and may be irradiated with NIR for 7 minutes. In at least one example embodiment, a fifth group may receive SPNC and may be irradiated with NIR for 1 minute. In at least one example embodiment, a sixth group may receive DOX.

Once tumor volumes reach or become greater than 100 mm³, the mice may be intratumorally injected with the various conditions (e.g., PLGA in the instance of the first group, SPNCD in the instance of the second group, SPNCD+NIR-1 minute in the instance of the third group, SPNCD+NIR-7 minute in the instances of the fourth group, SPNC+NIR-1 minute in the instance of the fifth group, and DOX in the instance of the sixth group) every 2 days for 15 days. Tumor size and mouse body weight may be measured every 2 days for 15 days in total. Bioluminescence imaging may be conducted by intraperitoneally-injected D luciferin (150 mg/kg) and imaging on an IVIS In Vivo Imaging System (e.g., PerkinElmer, USA) every 3 days for 15 days in total.

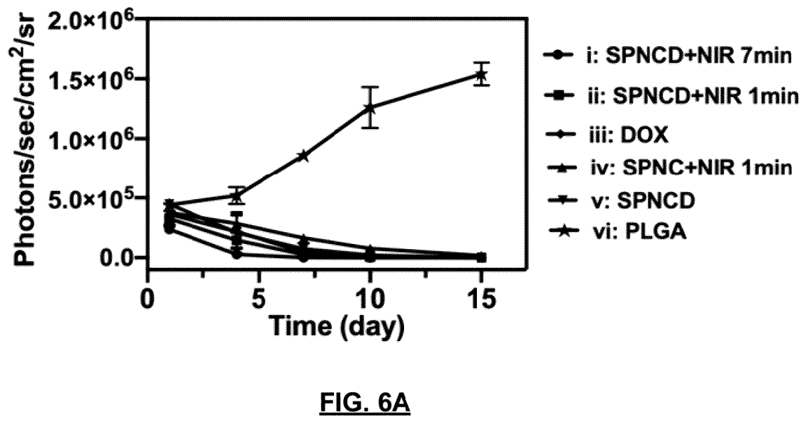

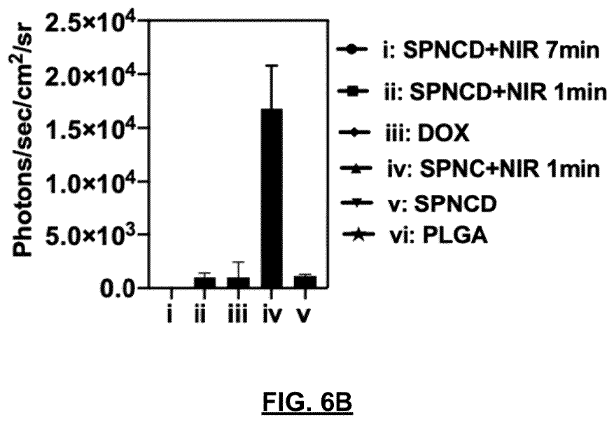

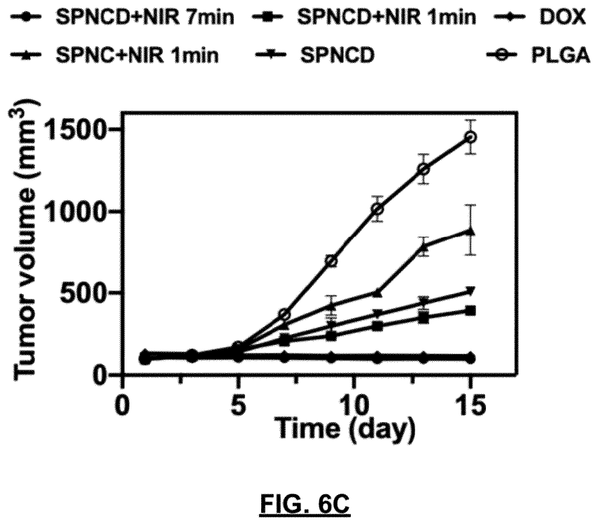

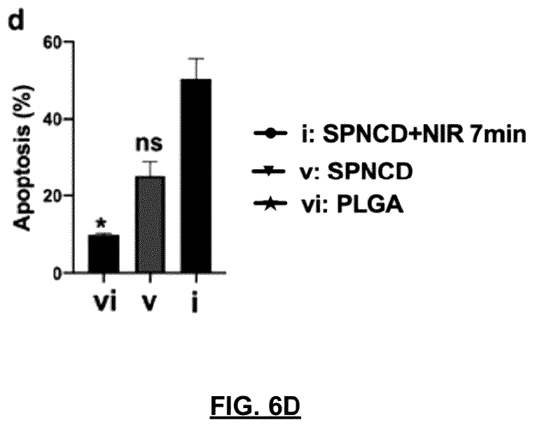

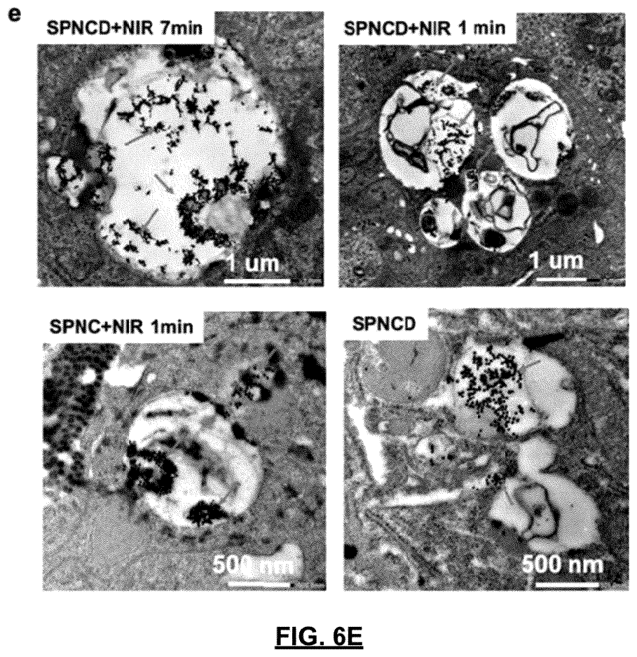

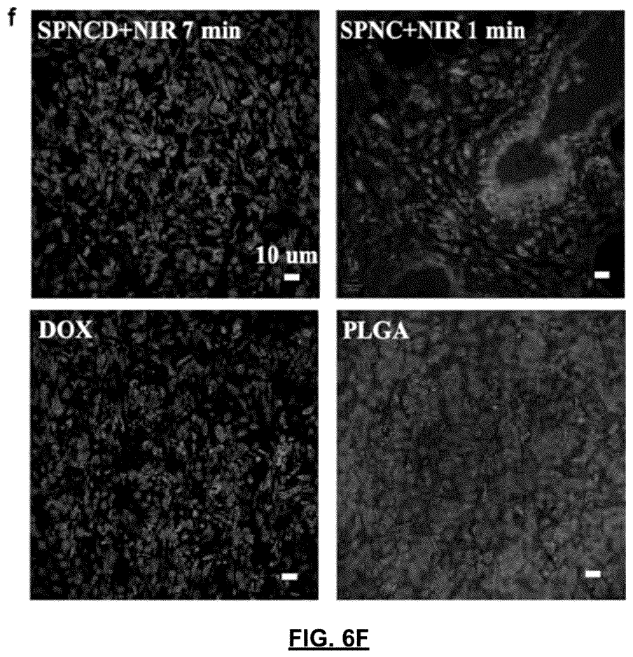

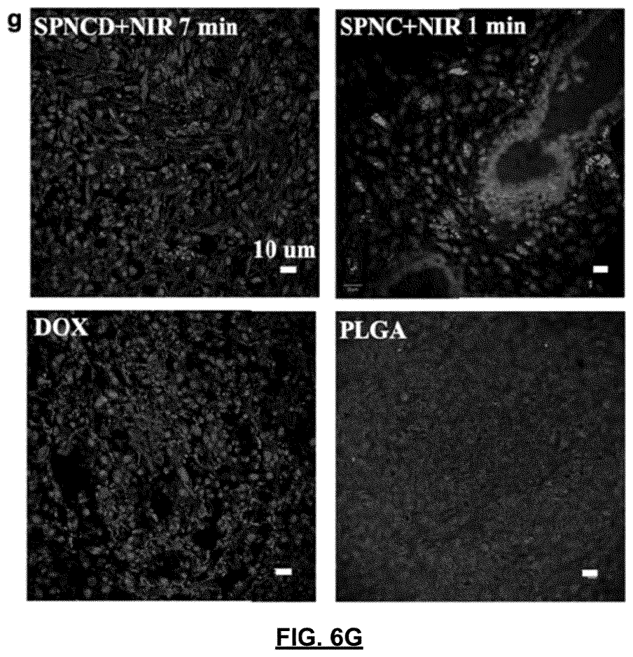

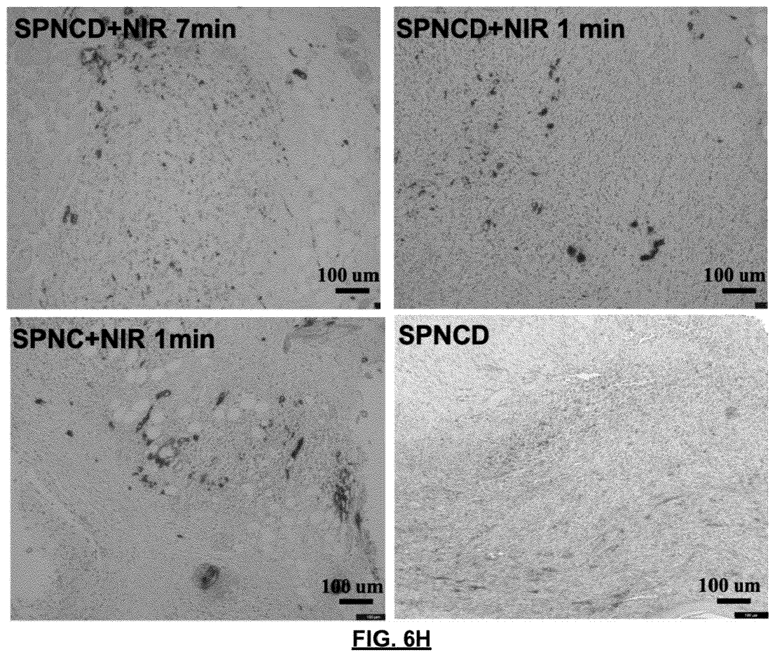

For example, FIG. 6A is a graphical demonstration illustrating bioluminescence signal intensity of the different samples, where the x-axis represents time in days and the y-axis represents photons in sec/cm²/sr; FIG. 6B is a fine detail bar graph of bioluminance signal intensity of the different samples on day 15 from FIG. 6A, where the y-axis represents photons in sec/cm²/sr; FIG. 6C is a graphical demonstration illustrating tumor growth in the mice receiving the different samples, where the x-axis represents time in days and the y-axis represents tumor volume in millimeters squared (mm²); FIG. 6D is a bar graph representing apoptosis in the mice receiving the different samples, where the y-axis represents apoptosis in percent; FIG. 6E are TEM images of tumor sections treated with the different samples; FIG. 6F are immunochemistry images of tumor sections treated with the different samples; FIG. 6G are Tunnel assays of tumor sections treated with the different samples; and FIG. 6H are Prussian blue staining of tumor sections treated with the different samples.

As illustrated, the PLGA control group experiences rapid increases in tumor BLI signal over the 15 days, representing rapid cell proliferation/tumor growth while the BLI signal from the SPNCD+NIR-7 minute group decreased sharply to effectively 0, significantly more than all other groups, including SPNCD alone and SPNCD+NIR-1 minute, suggesting that not only can the amount of DOX release (quantified by MPI) be increased by increasing NIR irradiation time, but that the rapid increase is linked to significant tumor cell killing effects in vivo. The BLI signal of SPNC+NIR 1min group is higher than the SPNCD+NIR 1min group, indicating that released DOX is the main driver of cell death, rather than the mild heat generated by NIR. Tumors in mice treated with SPNCD+NIR 7 min and DOX alone did not increase in size, indicating that these treatments kill or do not allow the cancer cells to proliferate. Flow cytometry and TUNNEL assays (see FIG. 6G) confirmed that both SPNCD+NIR 7 mins and DOX treatments induced significant apoptosis. As illustrated in FIG. 6E-6H, SPNCD (with/without NIR) completely degraded to release iron oxide nanoparticles by day 15 post-injection based on TEM and Prussian blue staining of tumor section. The data suggests that the observed MPI signal increase of each SPNCD+NIR group was due to increased Brownian relaxation rates upon release of iron oxide nanoparticles from the polymeric nanocomposite. Immunostaining (see FIG. 6F) showed that F4/80-positive TAM accumulation increased more in tumors treated with SPNCD+NIR 7 min, SPNCD+NIR 1min, SPNCD, SPNC+NIR 1min and DOX compared with PLGA treated groups

### Example 11

### Long-Term Tumor Inhibition and Metastasis

4T1 tumor-bearing Balb/c mice and BT549 tumor-bearing Nude mice ay be randomly divided into three groups. In at least one example embodiment, each group may include eight mice (PBS, SPNCD+NIR 7 min, DOX). Once tumor volumes reach or become greater than 100 mm³, 4T1 tumor-bearing mice (DOX: 0.122 mg/kg) and BT549 tumor-bearing mice (DOX: 1.5 mg/kg) may be intratumorally injected with agents from the various conditions every 2 days for 15 days followed by NIR irradiation (808 nm, 1W). In at least one example embodiment, the DOX dose may be determined based on the IC50 of each cell line. Tumor size and mouse weight may be measured every 3 days for 80 days in total. Bioluminescence imaging (e.g., PerkinElmer, USA) may be conducted every 3 days by intraperitoneally injected D luciferin (e.g., 150 mg/kg) into 4T1 tumor-bearing Balb/c mice to observe the metastasis of 4T1 tumor-bearing Balb/c mice.

### Example 12

### H&E, TUNNEL, and Immunohistochemistry Assay

Histology may be performed to assess DOX-related toxicity. To assess the histology of major organs (such as, the heart, liver, lung, spleen, and/or kidney) of the mice, in at least one example embodiment, all organs may be fixed with about 10% formalin, processed by H&E stain, and imaged with Digital Microscopy (e.g., Keyence VHX-6000).

To evaluate cellular apoptosis in tumors, tumors may be harvested and processed by staining the tumor slides with a TUNNEL assay apoptosis detection kit (e.g., catalog #: S7110, Sigma). Fluorescence images may be captured by Nikon A1Rsi confocal laser scanning microscopy (“CLSM”). In at least one example embodiment, FITC may be excited at 495 nanometers and collected at 519 nanometers. In at least one example embodiment, DAPI may be excited at 488 nanometers and collected at or between 580 nanometers and 630 nanometers.

To quantify macrophages in tumors, mice may be sacrificed on the last day (15^{th} day after the 1^{st} dose) of the short-term tumor inhibition experiment and the tumors may be harvested and processed via immunohistochemistry assay by staining tumor sections with F4/80 (e.g., Excitation: 647 nm; Emission: 660 nm) and DAPI (e.g., Excitation: 488 nm, Emission: 580-630 nm). Slides may be imaged using, for example, a Nikon A1Rsi CLSM.

### Example 13

### Iron Distribution in Tumors

To assess intratumoral SPNCD degradation, tumors may be sectioned with a diamond knife in order, collected onto 200 mesh copper grids, and sections may be imaged, for example, with TEM (e.g., JEOL 1400 Flash, USA).

To evaluate the distribution of iron in tumors on a larger scale, tumors may be processed with Prussian blue staining and imaged with Digital Microscopy (e.g., Keyence VHX-6000).

### Example 14

### Systematic Toxicity Evaluation

To evaluate systemic chemotherapy toxicity, 4T1 tumor bearing nude mice (Female, 6-8 weeks) may be put on a high fat diet (HFD) for two weeks pre-dosing, which maintained throughout the experiment, to mimic the human Western diet that can increase the sensitivity of the body to DOX. 4T1 tumor-bearing nude mice may be randomly divided into four groups when the tumor volume reached >100 mm³. In at least one example embodiment, each group may include five mice. In at least one example embodiment, the first group (Group 1) may receive 1.5 milligram (mg)/kilogram (kg) of DOX via IV injection. In at least one example embodiment, the second group (Group 2) may receive 1.5 milligram/kilogram of DOX via intratumoral injection. In at least one example embodiment, the third group (Group 3) may receive SPNCD+NIR 7 min (1.5 mg/kg) via intratumoral injection. In at least one example embodiment, the fourth group (Group 4) may receive vehicle control (PBS) via IV Injection.

In each instance, the mice may be dosed every three days and may receive a total of five doses. In at least one example embodiment, to assess blood biomarkers, greater than or equal to about 150 milliliters to less than or equal to about 200 milliliters of blood may be collected from retro-orbital sinus at 24 hours post injection of the fourth dose. In at least one example embodiment, greater than or equal to about 800 milliliters to less than or equal to about 1,000 milliliters of blood may be collected via cardiac puncture at 60 hours post injection of the fifth dose.

In at least one example embodiment, serum may be obtained by centrifuging the blood at 10,000 grams for 10 minutes at 4° C. Cardiotoxicity- and hepatotoxicity-related biomarkers may be evaluated using cTnl (e.g., ELH-CTNI-1, RayBiotech), LDH (e.g., ab102526, abcam), and/or ALT (e.g., ab105134, abcam) ELISA kits. Other major organs (such as heart, liver, lung, spleen, and/or kidney) may be evaluated for toxicity by processing with H&E staining, imaging with Digital Microscopy (e.g., Keyence VHX-6000) and through pathologists' evaluations.

### Example 15

### Tumor Therapeutic/Metastasis Evaluation

Tumor growth, mouse bodyweight, and tumor metastasis may be monitored for 80 days in 4T1 DOX-sensitive and BT459 DOX-resistant tumor mice. The mice may be randomly divided into four groups. In at least one example embodiment, each group may include five mice. In at least one example embodiment, the first group (Group 1) may receive 1.5 milligram (mg)/kilogram (kg) of DOX via IV injection. In at least one example embodiment, the second group (Group 2) may receive 1.5 milligram/kilogram of DOX via intratumoral injection. In at least one example embodiment, the third group (Group 3) may receive SPNCD+NIR 7 min (1.5 mg/kg) via intratumoral injection. In at least one example embodiment, the fourth group (Group 4) may receive vehicle control (PBS) via IV Injection.

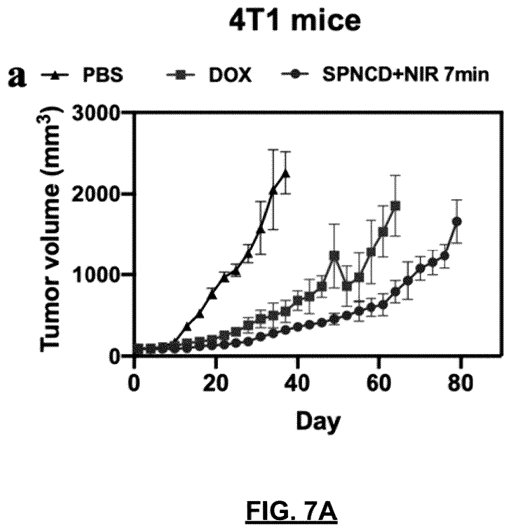

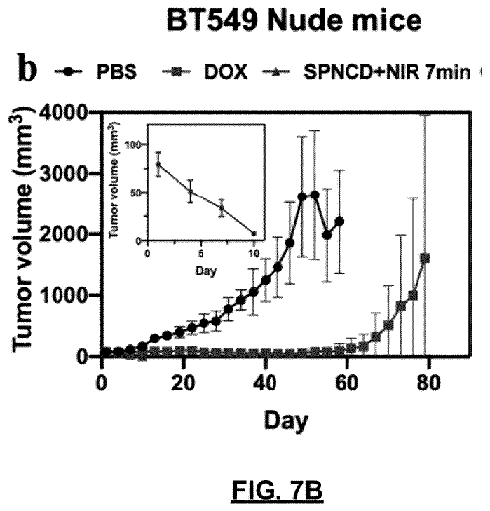

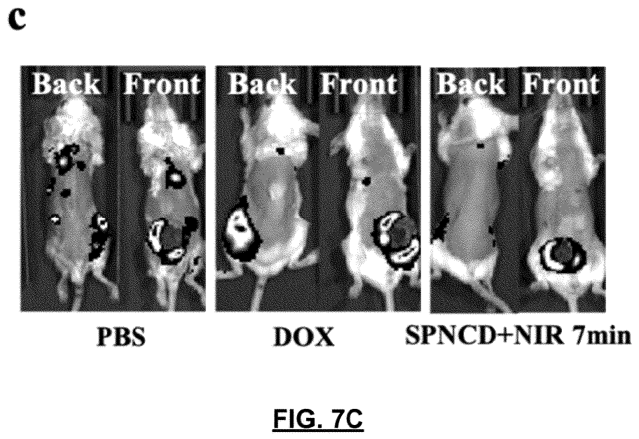

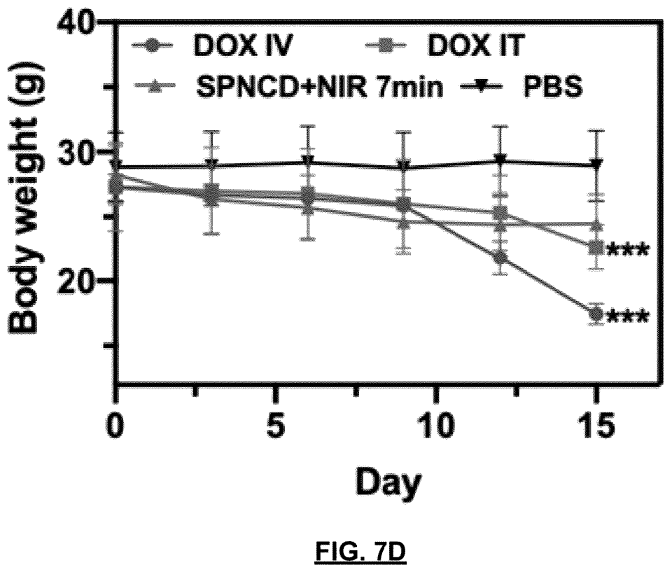

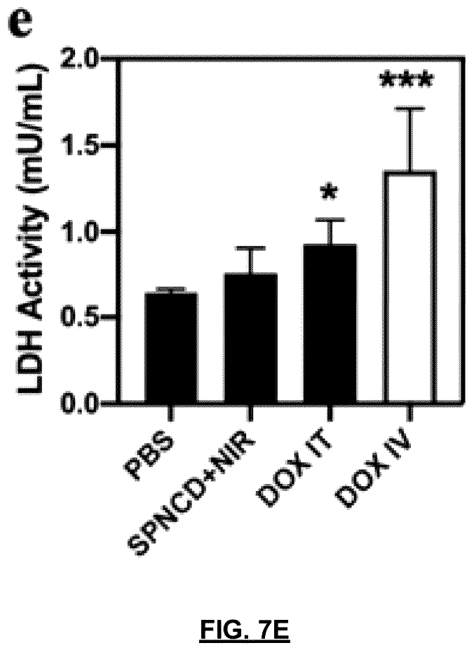

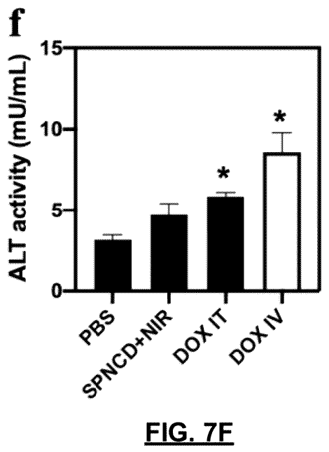

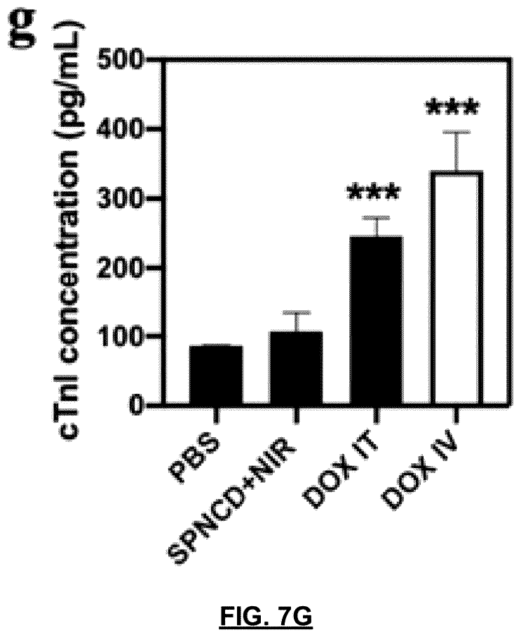

FIG. 7A is a graphical demonstration comparing tumor volumes for 4T1 mice treated with the different samples, where the x-axis represents day and the y-axis represents tumor volume in millimeters squared; FIG. 7B is a graphical demonstration comparing tumor volumes for BT549 mice treated with the different samples, where the x-axis represents day and the y-axis represents tumor volume in millimeters squared; FIG. 7C are BLI images of subjects receiving the different samples; FIG. 7D is a graphical demonstration comparing body weights of subjects treated with the different samples, where the x-axis represents day, and the y-axis represents body weight in grams (g); FIG. 7E is a bar graph comparing LDH activity of subjects treated with the different samples, where the y-axis represents LDH activity in mU/mL; FIG. 7F is a bar graph comparing ALT activity of subjects treated with the different samples, where the y-axis represents ALT activity in mU/ML; and FIG. 7G is a bar graph comparing cTnl concentrations of subjects treated with the different samples, where the y-axis represents cTnl concentration in pg/mL.

As illustrated, SPNCD+NIR-7 minute prolongs time to tumor recurrence and mouse survival compared with DOX alone and PBS treated mice. SPNCD+NIR-7 mins significantly inhibits 4T1 tumor growth (FIG. 7A) and prolongs the time-to-tumor metastasis (75 days post injection) compared with PBS vehicle (30 days post injection) and DOX alone (60 days post injection) control groups (FIG. 7C). In BT549 tumor mice, SPNCD+NIR-7 mins eliminated tumors by the fourth dose, with no recurrence (see FIG. 7B). The wound (caused by NIR irradiation) fully recovered on 32 days post injection and the mice remain alive (10 months after the first dose) with no tumor recurrence observed. The reason that SPNCD+NIR-7 minutes may work better on BT549 tumor bearing mice than 4T1 tumor bearing mice may be because the released drug alone is the therapeutic driving force on 4T1 tumor bearing mice, but both the released drug and PTT are the therapeutic driving force on BT549 tumor mice.

Treating the mice with 1.5 mg/kg (the same dose as the DOX-resistant tumor mice), we found that the bodyweight of SPNCD+NIR-7 minutes treated mice is significantly higher than the mice treated with DOX through both intravenous injection (IV) and intratumoral injection (IT) on day 15 (FIG. 7D), indicating increased tolerability and decreased toxicity. Further, SPNCD+NIR-7 minutes did not increase the expression level of cardiotoxicity related biomarkers (LDH, cTnl) and hepatotoxicity related biomarkers (ALT) (see FIG. 7E-7G), did not cause spleen contraction, did not generate significant cardiovacuolation, did not significantly shrink the size of white pulps and the number of red pulps compared with other control groups. SPNCD+NIR-7 minutes showed excellent efficacy, with tumors eliminated after two weeks treatment, with only a scar remaining where the tumor had been. The current nanoplatform significantly enhanced antitumor efficacy and even eliminate tumors on BT549 tumor bearing mice, while minimizing side effects on healthy organs.

### Example 16

### Statistical Analysis

In at least one example embodiment, statistical differences may be determined by one-way ANOVA and results may be expressed as means±SE. In at least one example embodiment, a p-value less than 0.05 may be considered to indicate statistical significance. In at least one example embodiment, statistical analyses may be performed using GraphPad Prism 7 (e.g., GraphPad Inc.).

The foregoing description of the embodiments has been provided for purposes of illustration and description. It is not intended to be exhaustive or to limit the disclosure. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the disclosure, and all such modifications are intended to be included within the scope of the disclosure.

1. A method for targeting and tuning in vivo drug release in a human or non-human subject, the method comprising:
monitoring release of one or more drugs from one or more nanocomposites administered to the subject, the monitoring including:
collecting information regarding the release of the one or more drugs from the one or more nanocomposites, and
analyzing the collected information to determine if a concentration of the released one or more drugs is within a therapeutic window; and
applying precision-controlled optical stimulus to an area of the subject hosting the one or more nanocomposites to accelerate release of the one or more drugs from the one or more nanocomposites when the concentration of the released one or more drugs is below the therapeutic window.

2. (canceled)

3. The method of claim 1, wherein the monitoring includes magnetic particle imaging, intensity of magnetic particle imaging signals varying in response to the release of the one or more drugs.

4. The method of claim 1, wherein the precision-controlled optical stimulus includes near-infrared light irradiation.

5. The method of claim 1, wherein the precision-controlled optical stimulus is applied for a period greater than or equal to about 1 minute to less than or equal to about 20 minutes.

6. The method of claim 1, wherein the one or more nanocomposites each includes a biodegradable polymer shell that at least partially encompasses a core that includes magnetic nanoparticles.

7. The method of claim 6, wherein the biodegradable polymer shell includes a polymeric material selected from the group consisting of: poly(lactide-co-glycolide acid) (PLGA), polymerized ursodeoxycholic acid (PUDCA), polyethylenimine (PEI), chitosan, poly(d-lactic acid), poly(s-caprolactone) (PCL), polylactic acid, polyanhydrides, polyphosphoesters, polyurethanes, poly(caprolactone), hyaluronic acid, albumin, polysaccharides, and combinations thereof.

8. The method of claim 6, wherein the magnetic nanoparticles include Fe₃O₄ nanoparticles.

9. The method of claim 6, wherein the one or more drugs are dispersed with the magnetic nanoparticles in the core.

10. The method of claim 6, wherein the one or more drugs are embedded in the biodegradable polymer shell.

11. The method of claim 6, wherein the one or more drugs includes doxorubicin.

12. The method of claim 6, wherein the method further includes preparing the one or more nanocomposites by contacting nanoclusters including the biodegradable polymer shell and the magnetic nanoparticles with the one or more drugs.

13. The method of claim 12, wherein the method further includes preparing the one or more nanoclusters by contacting the magnetic nanoparticles and the biodegradable polymer shell in a solvent and applying a mixing force.

14. The method of claim 1, wherein the method further includes administering the one or more nanocomposite to the subject.

15. A method for targeting and tuning in vivo drug release in a human or non-human subject, the method comprising:
collecting information regarding release of one or more drugs from one or more nanocomposites administered to a subject;
determining, using the collected information, if a concentration of the released one or more drugs is outside of a therapeutic window;
if the concentration of the released one or more drugs is within the therapeutic window, continuing to collect information regarding the release of the one or more drugs from the one or more nanocomposites; and
if the concentration of the released one or more drugs is below the therapeutic window, causing a precision-controlled optical stimulus to be applied to an area of the subject hosting the one or more nanocomposites for a period greater than or equal to about 1 minute to less than or equal to about 20 minutes to accelerate the release of the one or more drugs from the one or more nanocomposites.

16. The method of claim 15, wherein the collecting includes magnetic particle imaging, intensity of magnetic particle imaging signals varying in response to the release of the one or more drugs, and the precision-controlled optical stimulus including near-infrared light irradiation.

17. The method of claim 15, wherein the one or more nanocomposites each includes a biodegradable polymer shell that at least partially encompasses a core that includes magnetic nanoparticles, the one or more drugs dispersed with the magnetic nanoparticles within the core, embedded in the biodegradable polymer shell, or a combination thereof.

18. The method of claim 17, wherein the method further includes preparing the one or more nanocomposites by contacting nanoclusters including the biodegradable polymer shell and the magnetic nanoparticles with the one or more drugs.

19. The method of claim 15, wherein the method further includes administering the one or more nanocomposite to the subject.

20. A method for targeting and tuning in vivo drug release in a human or non-human subject, the method comprising:
collecting information regarding release of one or more drugs from one or more nanocomposites administered to a subject using magnetic particle imaging, intensity of magnetic particle imaging signals varying in response to the release of the one or more drugs;
determining, using the collected information, if a concentration of the released one or more drugs is below a therapeutic window;
if the concentration of the released one or more drugs is within the therapeutic window, continuing to collect information regarding the release of the one or more drugs from the one or more nanocomposites; and
if the concentration of the released one or more drugs is outside of the therapeutic window, using near-infrared light irradiation to accelerate the release of the one or more drugs from the one or more nanocomposites, the near-infrared light irradiation applied to an area of the subject hosting the one or more nanocomposites.
